(19) United States
(12) Patent Application Publication
(10) Pub. No.: US 2026/0248747 A1
(43) Pub. Date: Aug. 27, 2026

(54) PHYSICAL PERFORMANCE AID

(71) Applicant: aXichem AB, Malmö (SE)

(72) Inventors: Torsten HELSING, Kleppestø (NO); Lucas ALTEPOST, Nesøya (NO); Carlos Alix FAGES, Murcia (ES); Pablo Jiménez MARTINEZ, Albacete (ES)

(73) Assignee: aXichem AB, Malmö (SE)

(21) Appl. No.: 19/163,114

(22) PCT Filed: Mar. 12, 2024
(86) PCT No.: PCT/EP2024/056493

§ 371 Date: Sep. 8, 2025

(30) Foreign Application Priority Data
Mar. 13, 2023 (NO) 20230263

(51) Int. Cl. A61K 31/165; A61K 31/195; A61K 31/198; A61K 31/355; A61K 31/375; A61K 31/522; A61K 36/41; A61K 36/81; A61K 45/06; A61P 21/00

(52) U.S. Cl. CPC A61K 31/165; A61K 31/195; A61K 31/198; A61K 31/355; A61K 31/375; A61K 31/522; A61K 36/41; A61K 36/81; A61K 45/06; A61P 21/00

(57) ABSTRACT

The present invention relates to synthetic capsaicyns, tautomers or salts thereof for enhancing physical performance and/or reducing or delaying mechanical fatigue of a subject. More particularly, the invention provides compositions comprising synthetic capsaicyns of Formula (I), provided as sport diet supplements, use of the capsaicyn compounds or compositions for improving training performance, promoting accelerated recovery and/or reducing or delaying mechanical fatigue. Furthermore, compositions comprising capsaicyns for use in treatment of a disease or indication associated with physical weakness or a lack of energy, and methods for using the compounds and compositions, are provided.

### PRIOR RELATED APPLICATIONS

The present application is a national entry of PCT Application No. PCT/EP2024/056493, filed on Mar. 12, 2024, which claims priority to Norwegian Application No. NO 20230263, filed on Mar. 13, 2023. The entire contents of such prior applications are incorporated by reference herein.

### FIELD OF THE INVENTION

The present invention relates to synthetic capsaicyns and compositions comprising such capsaicyns for enhancing physical performance and/or reducing or delaying mechanical fatigue of a subject. More particularly, the invention provides compositions comprising synthetic capsaicyns of Formula (I), provided as sport diet supplements, use of the compounds or compositions for improving training performance, promoting accelerated recovery and/or reducing or delaying mechanical fatigue of a subject. Furthermore, compositions comprising capsaicyns for use in treatment of a disease or indication associated with physical weakness or a lack of energy, and methods for using the compounds and compositions, are provided.

### BACKGROUND OF THE INVENTION

Sport supplements are popular ergogenic aids among athletes with the aim of maximize their performance in sport tasks. Recent sport nutrition research has focused on the discovery of new active compounds which may be able to improve high-intensity training in different modalities. Accordingly, research related to the effects of capsaicinoids in resistance training have grown in the last years, as shown by de Freitas et al. Acute Capsaicin Supplementation Improves Resistance Training Performance in Trained Men. J Strength Cond Res. 2018; 32:2227-32. Capsaicinoids are a group of compounds naturally found in spicy chili peppers which are characterized by their vanilloid structure. Capsaicin (8-methyl-N-vanillyl-trans-6-nonenamide), which is found in the placental tissue of Capsicum fruit has emerged as the primary and most abundant capsaicinoid with therapeutical and physical performance relevance.

As a transient receptor vanilloid 1 (TRPV1) agonist, plausible resistance training ergogenic mechanisms underlying capsaicin are an increment in the calcium released by sarcoplasmic reticulum and a heat-analgesic effect. Accordingly, in high-intensity tasks, afferents III and IV nervous fibers are linked to peripheral and central fatigue. These small-diameter muscle afferents contribute to the perceptual responses during exercise which may lead to discomfort, an increase in rating of perceived exertion (RPE) and neuromuscular fatigue. As these fibers directly or indirectly reduce motoneuron firing and motor unit recruitment, their TRPV1 content (mainly in group 4 afferents) has been proposed as a physical performance target. On the other hand, although capsaicin TRPV1 activity may be linked to its distinctive spiciness, encapsulated capsaicin has not got a direct pungent taste. However, previous research, Opheim M N, Rankin J W. Effect of Capsaicin Supplementation on Repeated Sprinting Performance. J Strength Cond Res. 2012; 26:319-26, has reported intestinal discomfort after high-dose oral encapsulated-capsaicin supplementation (25.8 mg).

To date, capsaicinoids supplementation approach on sport performance have been tested under oral capsaicin supplementation, please see Jiménez-Martínez P. et al. Effects of oral capsaicinoids and capsinoids supplementation on resistance and high intensity interval training: A systematic review of randomized controlled trials. J HumSportExer. 2022. Purified acute (i.e., 45 minutes prior to exercise) oral capsaicin supplementation have elicited performance improvements in upper and lower-limbs resistance training tasks. Hence, de Freitas et al. showed in a double blind, randomized placebo-controlled trial that acute 12 mg capsaicin supplementation increased significantly the number of repetitions until failure, the total weight lifted and reduced rated perceived exertion overall body (RPE-OB) in squat exercise with a not-volume-matched design. By contrast, a lower dose of purified capsaicin (i.e., 1.2 mg in gummy format) did not reach significant differences in summed torque or fatigue index of an isokinetic knee extension exercise in a randomized, double blinded, controlled trial, as shown by Cross B L, et al. Effect of a Commercially Available Low-Dose Capsaicin Supplement on Knee Extensor Contractile Function. 2020; 7. Besides, although velocity-based training has been proposed as an objective approach to resistance training by reflecting the state of the neuromuscular system to produce force against a load, only one study, da Silva BVC, et al. Acute Supplementation with Capsaicin Enhances Upper-Limb Performance in Male Jiu-Jitsu Athletes. Sports. 2022; 10:120, have elicited the effects of capsaicin on velocity variables in resistance training. Furthermore, research approaching capsaicinoids effects on velocity-derived outcomes have only been reported for upper limbs exercise but not for any lower limbs activity (i.e., squat exercise), ref. da Silva, and Jiménez-Martínez P.

Accordingly, there is a need for alternative compounds and compositions for use as sport supplements and for positively affecting sport performance and/or reducing or delaying mechanical fatigue of a subject.

### BRIEF DESCRIPTION OF THE DRAWINGS

FIG. 1 shows results from the study of example 1 evaluating the ergogenic effects of phenylcapsaicyn (PC), providing individual (points) and mean (bars) values of:
(a) RPE-OB, Overall body rating of perceived exertion;
(b) RPE-AM, Active muscle rating of perceived exertion;
(c) PRS, Perceived recovery status, for three different supplementation conditions.

FIG. 2 shows results from the study of example 2 evaluating the ergogenic effects of phenylcapsaicyn (PC), providing individual (points) and mean (bars) values of
(a) CMJ height at pre-test, post-test and CMJ post 24 h,
(b) CMJ pre-post-test percentage of difference and
(c) CMJ pre-post 24 h percentage of difference, for three different supplementation conditions.

FIG. 3 provides the mean propulsive velocity (MPV) in an intra-set comparison of individual repetitions and repetitions across time for the different supplementation conditions (PLA, HD, LD) of phenylcapsaicyn (PC), of example 2.

### BRIEF SUMMARY OF THE INVENTION

The inventors have discovered that a group of synthetic capsaicyns have an ergogenic effect, and that supplementation of such capsaicyns prior to physical exercise may increase performance, promote accelerated recovery post-exercise and reduce or delay mechanical fatigue.

The invention provides a composition comprising a compound of formula (I) or tautomers or salts thereof

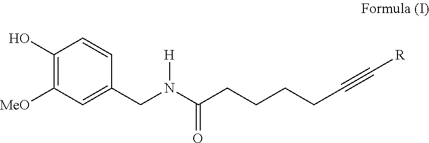

wherein R is alkyl, trifluoromethyl, cycloalkyl, phenyl, or halogen, and when the substituent R comprises a carbon chain, it is straight-chained or branched and optionally further substituted with alkyl, alkenyl, alkynyl, allyl, aryl, alkoxy, aryloxy, alkanoyl, aroyl, amino alkylthio, arylthio, cyano, cycloalkyl, cycloalkenyl, halo, hydroxy, oxo, nitro, or trifluoromethyl,
   for enhancing physical performance, promoting accelerated recovery and/or reducing or delaying mechanical fatigue of a subject.

In another aspect, the invention provides a composition e.g. formulated as a sport diet supplement comprising a compound of formula (I), e.g. provided in a unit dosage form of 0.5 to 4.0 mg of a compound of Formula I.

In yet another aspect the invention relates to use of a compound of formula (I), for enhancing physical performance during an exercise and/or reducing or delaying mechanical fatigue of subject, wherein the compound is administered to the subject, such as prior to the exercise. The compound of Formula (I) may be used in a composition.

In yet another aspect, the invention relates to a composition comprising a compound of formula (I), for use in treatment of a subject suffering or recovering from a disease or indication associated with physical weakness or a lack of energy such as chronic fatigue syndrome (CFS), myalgic encephalomyelitis (ME) or ME/CFS.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

There is a need for nutritional compositions capable of improving high-intensity training and increasing mechanical performance, such as to favourably impact training performance, the perceived exertion of an exercise, muscle damage, protein breakdown, perceived fatigue, and recovery after training.

In the last years, synthetic analogues including phenylcapsaicyn (PC) have emerged as a plausible alternative to traditional oral purified capsaicin supplementation, see EFSA Panel on Nutrition, Novel Foods and Food Allergens (EFSA NDA Panel), Turck D et al. Safety of phenylcapsaicin as a novel food pursuant to Regulation (EU) 2015/2283. EFSA J [Internet]. 2019 [cited 2022 Sep. 14].

The applicant has surprisingly found that certain synthetically produced capsaicin analogues, including phenylcapsaicyn, can positively affect a subject's sport performance, promote accelerated recovery post-exercise, including also reducing or delaying mechanical fatigue.

The present invention hence relates to synthetic compounds, and compositions comprising the compounds, that are safe and effective to maintain or enhance sport performance in mammals in general, and in particular, in human subjects. The compounds or compositions can advantageously be used alone or in a combination with any diet and/or therapy to support, re-establish or enhance the training performance and endurance in human subjects. Accordingly, the invention in general relates to compositions that are effective to maintain or enhance physical performance in human subjects.

Compositions of the invention comprise at least one chemical compound of Formula (I)

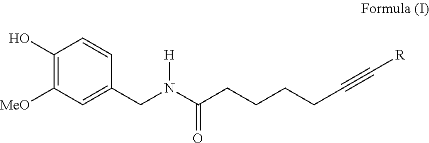

wherein R is alkyl, trifluoromethyl, cycloalkyl, phenyl, or halogen, or tautomers or salts thereof.

When said substituent R comprises a carbon chain, it is straight-chained or branched and optionally further substituted with alkyl, alkenyl, alkynyl, allyl, aryl, alkoxy, aryloxy, alkanoyl, aroyl, amino alkylthio, arylthio, cyano, cycloalkyl, cycloalkenyl, halo, hydroxy, oxo, nitro, or trifluoromethyl.

When said R comprises a carbon chain, the carbon chain may preferably be alkyl. The carbon chain may have a 1 to 6 carbon atom long chain, more preferably a 1 to 4 carbon atom long chain. More preferably, R may be isopropyl, a C4 alkyl, or preferably phenyl. The compound wherein R is phenyl is thus termed phenylcapsaicyn.

In one embodiment, the compound of Formula I is selected from the group of methylcapsaicyn, ethylcapsaicyn, a propylcapsaicyn, a butylcapsaicyn, and phenylcapsaicyn. In one embodiment, the compound is phenylcapsaicyn.

The compound of Formula (I) is herewith termed R-capsaicyn. It is important to note the difference in structure between capsaicin, a pepper analogue, and R-capsaicyns. Firstly, capsaicin contains a double bond instead of a triple bond, which is present in R-capsaicyn. Hence, the difference in the endings -in and -yn. Said substituent R is therefore not a substituent at the end of a capsaicin molecule, but at the end of an analogue, which may have different properties from capsaicin. EP patent 1 670 310 B1 of the applicant discloses how R-capsaicyns can be synthesised and produced, thus avoiding the limitations and disadvantages of extracting the compounds from natural products and raw materials.

The invention hence provides a compound of Formula (I), compositions comprising this, and the use of this as an ergogenic aid and thereby enhance physical performance, stamina and/or reduce perceived fatigue.

Compositions of the invention can be advantageously used for enhancing physical performance, promoting accelerated recovery and/or reducing or delaying mechanical fatigue. In one aspect, the invention provides a compound of Formula (I), for enhancing physical performance and/or reducing or delaying mechanical fatigue. Alternatively, it is provided the use of a compound of Formula I, for enhancing physical performance, accelerate recovery and/or reducing or delaying mechanical fatigue of a subject, wherein the compound is administered to the subject. Preferably the compound of Formula (I) is included in a composition formulated for administration to a human subject.

The current invention is partly based on findings from studies performed by the applicant. Two such studies, as detailed in Example 1 and 2, provide evaluations of the ergogenic effect of phenylcapsaicyn in two different doses. Although velocity-based training has been proposed as an objective approach to resistance training by reflecting the state of the neuromuscular system to produce force against a load, only one earlier study by da Silva BVC et al, have elicited the effects of capsaicin on velocity variables in resistance training. Furthermore, as mentioned, capsaicinoids effects on velocity-derived outcomes have only been reported for upper limbs exercise but not for any lower limbs activity (i.e., squat exercise). Acute muscle damage, protein breakdown and recovery variables have not earlier been measured after capsaicin supplementation. As intense exercise may increase metabolic, biochemical and neuromuscular fatigue in acute and chronic ways, exploring the biochemical, perceptual and neuromuscular effects of capsaicin and capsaicinoids on these topics may also be relevant. Therefore, the aim of this study was to explore the effects of a synthetic capsaicyns on lower limbs performance in the squat exercise under velocity-based control, metabolic responses to exercise, acute biochemical muscle damage and protein breakdown, rated perceived exertion for the overall body (RPE-OB) and for the active muscle, and the perceived recovery in resistance trained men.

The studies provide evidence-backed ergogenic effect of phenylcapsaicyn. More particularly, example 1 supports that compounds of Formula I, when administered prior to exercise, may increase performance, reduce muscle damage, reduce protein breakdown, and reduce perceived exertion and recovery time. Example 2 supports that compounds of Formula I exert a positive effect on mechanical dynamic performance, when administered prior to exercise, may increase neural activity during dynamic resistance training and may reduce acute mechanical fatigue. Phenylcapsaicyn ingestion may be considered as an anti-fatigue ergogenic aid and mechanical fatigue after exercise may be delayed.

The exercise, which the performance may be enhanced for, is typically a physical training, and particularly a strenuous physical activity, such as a high-intensity training, a resistance training, velocity-based training, high-volume training, dynamic resistance training, or interval training.

The capsaicyns of Formula I, and the compositions comprising such, elicit physical performance by affecting, or being manifested as, any one or more of:
Resistance training performance: e.g. increase performance, such as the number of repetitions before failure, total weight lifted, force or power production. e.g. as measured in a squat exercise, velocity-load assessment, Repetition Maximum (RM);
Mechanical dynamic performance: e.g. increase isometric and/or dynamic performance, such as positively affecting force production, velocity-derived outcomes as velocity variables like velocity of a movement, velocity loss of movement, distance of movement, e.g. as measured in a squat exercise, countermovement jump (CMJ) and isometric squat.
Muscle damage: reduce biomedical muscle damage, e.g. by measured aspartate aminotransferase (AST) levels in blood of the subject, e.g. as a lower level of AST post training, such as 24 hours post training, compared to subjects having received placebo;
Metabolic response: e.g. by lowering post-test lactate blood levels, which may be measured as a metabolic indicator of the exercise intensity;
Protein breakdown: e.g. by lowering the level of urea in blood as a measure of whole-body protein breakdown.
Neuromuscular fatigue: mean propulsive power, mean propulsive force and velocity loss may be measured as indicators or neuromuscular fatigue, e.g. in a squat exercise.
Neural excitability, and recovery; Improve recovery outcome, shorten recovery time, particularly mechanical recovery outcomes.
Affecting neuromuscular activity, delaying effect on neuromuscular fatigue after training, reduce perceived fatigue, such as attenuate acute and/or post-exercise mechanical fatigue.

Accordingly, the use of a compound of Formula I, or a composition comprising this, enhances physical performance positively affecting either of resistance training performance or mechanical dynamic performance, muscle damage, metabolic response, protein breakdown, neuromuscular fatigue, neural excitability or neuromuscular activity, including accelerate recovery and/or reduce recovery time.

More specifically, the following in vivo parameters may be affected:
Lactate Levels: Indicator of metabolic stress and intensity of exercise.
Urea Levels: Reflective of protein breakdown and muscle metabolism.
Aspartate Aminotransferase (AST) Levels: As a marker for muscle damage and recovery post-exercise.

In one embodiment, the use of the compound of Formula I, or composition comprising this, provides one or more of lowering the level of aspartate aminotransferase (AST), lowering the level of urea or lowering the level of lactate in the subject's blood, and thereby enhancing physical performance during exercise and/or reducing or delaying mechanical fatigue after exercise. As detailed in Examples 1 and 2, the supplementation of the compound phenylcapsaicyn, in two different dosages, prior to exercise lowered the subject's blood level of AST, urea and lactate post-exercise, and thereby enhanced physical performance.

The discovery of these previously unknown properties of the compounds of Formula I, providing the new technical effect recited above, clearly involves a valuable and inventive contribution to the art.

In one embodiment, the effect is manifested as either of a reduced perceived exertion for the active muscle, enhanced mechanical performance, lower muscle damage or reduced protein breakdown. Furthermore, the effect may be is manifested as an increased velocity, force and/or power production, and particularly as an increased velocity, e.g. measured as mean propulsive velocity (MPV). In one embodiment, the improved performance is in the form of improved force and/or power production.

In relation to the first study, Example 1, it was hypothesized that capsaicyn of Formula I may exert a positive impact in a dose-response way on velocity outcomes and rated perceived exertion (RPE), and this was indeed found. However, a concomitant increment of perceived fatigue, protein breakdown and muscle damage was also expected due to the increment in physical performance after the supplementation. It was expected that impaired recovery and a higher muscle and protein damage were higher when supplemented with a higher dose. Surprisingly, the results of the study suggest that a high dose (2.5 mg) of phenylcapsaicyn supplementation ingested 45 minutes prior to exercise may indeed increase performance, but also reduce muscle damage (lowered AST), reduce protein breakdown (lowered urea), reduce peripheral perceived exertion, showing mechanical anti-fatigue effects on trained subjects. Subjective fatigue and recovery assessment was conducted using the perceived recovery status, and as indicated in FIG. 1 c) the compounds of Formula I may also promote accelerated recovery post-exercise. Hence, these new findings are valuable at least because never before has a capsaicinoid been evaluated concerning muscle damage, protein breakdown, and peripheral fatigue.

Human subjects according to invention may be healthy physically active individuals, or alternatively individuals suffering of or recovering from a disease. By “healthy physically active individual” in the present context is meant a human subject who has a good physical health, i.e. an individual that has his/her bodily functions and processes working normally, not necessarily at their peak, but not significantly deviating from their peak to affect individual complete physical, mental, and social well-being, in particular bodily functions and processes relating to musculoskeletal activity. In one preferred embodiment, a healthy physically active individual is a human subject who has regular periods of intensive physical activity requiring significant energy expenditure, e.g. a subject who is doing physical exercise to improve his/her physical performance such as a sportsman. The use of compounds of Formula (I) or compositions comprising this can help such individuals to restore, maintain and/or enhance the physical performance and/or reduce or delay the perceived fatigue of an exercise. The condition of fatigue induced by the performance of exercises is a transitory physiological condition caused by natural circumstances and removable by simple rest. Simple training is generally known as retarding the perception of fatigue. The use of compounds of Formula (1) results in enhanced performance during exercise and/or reduced or delayed fatigue, which are physiological conditions caused by natural circumstances. Accordingly, the preferred use of the compounds of Formula (I) is non-therapeutic.

In one embodiment, the human subject according to the invention is an individual suffering or recovering from a disease or has signs associated with acute or chronic medical conditions associated with physical weakness or a lack of energy. Examples of relevant diseases are chronic fatigue syndrome (CFS), also called myalgic encephalomyelitis (ME) or ME/CFS. In one embodiment, the condition is asthenia. CFS is a complex condition characterized by persistent and debilitating fatigue that is not improved by rest and may be worsened by physical or mental exertion. Patients with CFS/ME often experience a range of symptoms, including muscle pain, sleep disturbances, cognitive impairment, and post-exertional malaise, which is a worsening of symptoms after physical or mental activity. Currently, there is no specific medication that can cure CFS/ME, but some treatments may help alleviate symptoms and improve quality of life. For medical treatment, herein is provided a composition comprising at least a compound of Formula (I) for use in treatment of a disease selected from the group of chronic fatigue syndrome (CFS), myalgic encephalomyelitis (ME) and ME/CFS, wherein the composition is administered to a subject suffering or recovering from the disease.

One potential approach to helping patients with CFS/ME is to improve their physical performance and recovery. In one embodiment of the invention, the composition of the invention is for subjects with CFS. Generally, treatment can be achieved through the use of medications that target metabolic pathways in the body. For example, some medications that have been used to treat metabolic disorders such as type 2 diabetes or mitochondrial disorders have been shown to improve physical performance and recovery in some patients. One such medication is metformin, which is commonly used to treat type 2 diabetes. Metformin works by reducing the amount of glucose produced by the liver and improving insulin sensitivity, leading to improved energy metabolism. In some studies, metformin has been shown to improve physical performance in healthy individuals and in people with metabolic disorders. Another medication that may be helpful for patients with CFS/ME is coenzyme Q10 (CoQ10). CoQ10 is a naturally occurring substance that plays a key role in energy production in the body. Studies have shown that CoQ10 supplementation can improve physical performance and reduce symptoms in patients with metabolic disorders, and some evidence suggests that it may also be beneficial for patients with CFS/ME. Additionally, medications that improve mitochondrial function, such as nicotinamide adenine dinucleotide (NAD+) precursors, may also be helpful for patients with CFS/ME. NAD+ plays a crucial role in energy production in the body and is involved in many metabolic pathways. Some studies have shown that NAD+ precursors can improve mitochondrial function and physical performance in healthy individuals and in people with metabolic disorders. Medications that improve physical performance and recovery, such as the compositions of the invention, may hold promise for improving symptoms and quality of life in patients with CFS/ME.

The compound of Formula (I) may be included in the compositions in concentrations providing the disclosed effect. The concentration of the compound of Formula (I), as provided by parts per million (ppm), is such as, but not limited to: 1-500 ppm, 5-250 ppm, 10-100 ppm, 10-75 ppm, 10-50 ppm, 5-50 ppm, 1-50 ppm. It is routine work to select suitable amounts to be incorporated into said compositions. A skilled person is able to do so without undue burden. In one embodiment, the composition of the invention comprises a compound of Formula I, e.g. phenylcapsaicyn, in a concentration of 70.0-100 wt %, such as 90.0-100 wt %, preferably 97.0-100% of the composition. In the study of Example 1, a composition comprising 98 wt % phenylcapsaicyn was used.

The composition may further include one or more of any conventional, acceptable excipients and/or carriers, e.g. solvents, fillers, diluents, binders, lubricants, glidants, viscosity modifiers, surfactants, dispersing agents, disintegration agents, emulsifying agents, wetting agents, suspending agents, thickeners, buffers, pH modifiers, absorption-delaying agents, stabilisers, antioxidants, preservatives, antimicrobial agents, antibacterial agents, antifungal agents, chelating agents, adjuvants, sweeteners, aromas, and colouring agents. Conventional formulation techniques known in the art, e.g., conventional mixing, dissolving, suspending, granulating, drageemaking, levigating, emulsifying, encapsulating, entrapping or compressing processes, may be used to formulate the composition.

In one embodiment, the composition comprises excipients, e.g. to encapusulate the capsaicyn. As one example, the compound of Formula I, may be encapsulated to be provided in the form of microencapsulated capsaicyn, e.g. using an encapsulating material, e.g. cellulose and/or lipidic excipients, as primary vesicles. Microencapsulation might entail a lower pungency power, less digestive system mucosa irritation and potentially a higher bioavailability. In one example, the composition is provided in the form of encapsulated compound of Formula I, comprising excipients in a concentration of 0-3.0 wt %, such as 0.5-2.0 wt %.

Compositions of the invention may be formulated as supplements or alternatively as pharmaceutical compositions, depending on the use and the recipient. These formulations will comprise effective amounts of the essential ingredients of the composition of the invention in an appropriate molar ratio. The formulations can be prepared according to standard rules and proceeding established in the corresponding art. Said supplement comprises, but is not limited to, at least one of: dietary supplement, nutritional supplement, nutraceutical supplement, over-the-counter supplement and/or pharmaceutical grade supplement.

In one aspect, the invention provides a new composition, e.g. in the form of a sport diet supplement comprising a compound of formula I as disclosed above. The composition may be formulated as a powder, granulate, tablet, capsule, an aerosol inhalation formulation, or as a drink or beverage. In one embodiment, the composition is a nutritional supplement. The composition in the form of a powder or granulate may further be included or used in a drink, or a food, such as a bar. In one embodiment the composition is ready formulated in the form of a sport food or a sport beverage, such as a sport drink, an energy drink, a sport bar, or an energy bar. In one embodiment, the composition is formulated as a sport diet supplement.

Furthermore, in addition to the compound of Formula (I), the composition may further comprise one or more ingredients that synergize with the compound of Formula (I), e.g. phenylcapsaicyn, to enhance its effects on physical performance and recovery. In one embodiment, the one or more ingredients are selected from the group of adaptogens, amino acids, antioxidants, electrolytes, energy boosters and creatine.

Examples of more specific ingredients that may be included in the composition are: Adaptogens: Rhodiola Rosea or Ashwagandha, which can help improve endurance and recovery.
Amino acids: Specifically Branched-Chain Amino Acids (BCAAs) or L-arginine, to support muscle recovery and nitric oxide production.
Antioxidants: Vitamins C and E, or compounds like quercetin, to reduce oxidative stress and support recovery.
Electrolytes: Potassium, magnesium, and sodium for hydration and muscle function.
Energy Boosters: Natural caffeine from green tea or guarana for increased energy and focus.
Creatine: Enhance the composition's ability to improve physical performance, muscle strength, and recovery.

In one embodiment, the composition is a nutritional supplement, such as a sport diet supplement, comprising a compound of Formula (I), and further comprising one or more ingredients selected from the group of adaptogens, amino acids, antioxidants, electrolytes, energy boosters and creatine.

Preferably, the route of administration(s) is oral, sublingual, by inhalation, enteral and/or rectal. More preferably, the route of administration is oral. An advantage of oral administration is the low level of invasiveness, causing less stress in the subject than more invasive administration routes, such as parenteral. In some embodiments, the compound of Formula (I) or composition comprising this, is administered orally. In some embodiments, this is administered with a meal or before a meal. The compound or composition is formulated accordingly, preferably for oral administration, or alternatively for inhalation.

The applicant has found that capsaicyns of Formula (I) exert a positive effect on performance of a subject, by administering this prior to physical activity, in tolerable safe doses. This is in comparison to previous studies wherein natural capsaicin (8-methyl-N-vanillyl-trans-6-nonenamide) has been used, such as the one by de Freitas et al, who showed limited effect even though a dose of as high as 12 mg was used. Hence, an advantage of said composition is a reduction of the required amount of active substance for achieving the desired outcome compared to the required amount in the absence of said composition.

The compound or composition will according to the invention be administered to a subject in an effective dose. As used herein, the term “effective dose” means the amount of compound according to the invention which is effective for producing the desired effect on performance. The effective dosage amount may vary depending upon the route of administration and dosage form. Appropriate dosages may depend on the compound to be used, the stage of the condition, age and weight of the patient, etc. and may be routinely determined by the skilled practitioner according to principles well known in the art. A suitable daily dosage of the compound according to the invention may range from about 0.01 mg/kg body weight to 1.0 mg/kg body weight. For example, in some embodiments, the daily dose may be 0.01-0.1 mg/kg body weight, such as 0.01-0.05 mg/kg body weight such as 0.03-0.08 mg/kg body weight, such as 0.05-0.1 mg/kg body weight. In other embodiments, the daily dose may be 0.05-0.5 mg/kg body weight, such as 0.05-0.02 mg/kg body weight, such as 0.08-0.5 mg/kg body weight. In yet other embodiments, the daily dose, or single dose used in relation to exercise, may be 0.5 mg-4.0 mg, e.g. 0.625 mg, or 2.5 mg. In one embodiment, the compound of Formula I is provided in a composition presented in unit dosage form as a single dose, e.g. comprising 0.5-4.0 mg of a compound of Formula I, such as comprising 0.5-2.5 mg of a compound of Formula I, e.g. in a dosage of 0.5 mg, 0.625 mg, 1.0 mg, 1.5 mg, 2.0 mg, 2.5 mg, 3.0 mg, 3.5 mg, or 4.0 mg of a compound of Formula I.

For an optimal effect, the compound of Formula I, or the composition comprising this, should be administered to the subject prior to physical activity, and not too long before this. In one embodiment, the subject should be administered the compound or composition up to 60 minutes prior to exercise, such as 10-60 minutes prior to exercise, most preferably 30-50 minutes prior to exercise. In one embodiment, the subject ingests the compound or composition, such as by oral ingestion. The subsequent performance of the exercise is enhanced as compared with a physical performance of the subject during physical exercise in the absence of administration of compound I.

In another aspect, the invention provides use of a composition comprising a compound of formula (I) as disclosed above, in a human subject, thereby enhancing physical performance during an exercise and/or reducing or delaying mechanical fatigue, wherein the composition is administered to the subject prior to the exercise.

In yet another aspect, the invention provides a method for non-therapeutically enhancing physical performance and/or reducing or delaying mechanical fatigue of a subject, wherein a composition comprising a compound of formula (I) is administered prior to exercise.

In yet another aspect, the invention provides a composition comprising at least a compound of Formula (I) as disclosed for use in medical treatment of a subject suffering or recovering from a disease or indication associated with physical weakness or a lack of energy such as chronic fatigue syndrome (CFS), myalgic encephalomyelitis (ME) or ME/CFS, the use comprising administering a compound of formula (I), or a composition comprising a compound of Formula (I), to the subject. Alternatively, for non-therapeutic use, compositions of the invention may for instance be advantageously used by subjects recovering from a disease or condition, or subjects affected by a physical injury or environmental or psychological stress factors. In one embodiment, the use is for treatment of asthenia, such as for improving symptoms and quality of life of subjects having either of these indications, e.g. by improving physical performance, recovery or reduce or delay fatigue. In one embodiment, the use is for preventing or reducing asthenia. Likewise, the invention provides a method for treatment of a subject suffering or recovering from a disease or indications associated with physical weakness or a lack of energy, such as chronic fatigue syndrome (CFS), myalgic encephalomyelitis (ME) or ME/CFS, or asthenia, wherein the use enhances performance and/or reduces or delays mechanical fatigue, wherein a composition comprising a compound of formula (I) is administered to the subject.

The skilled person is familiar with how to produce and manufacture suitable compositions in accordance with good manufacturing practice (GMP). The skilled person is able to handle standard processes for producing stable Formulations and compositions.

The embodiments and features described in the context of one aspect, e.g. for the aspect directed to the compound of Formula (I) or the composition comprising such, also apply to the other aspects of the invention, such as in the use thereof or in a method. The varieties of compounds and active substance options, and combinations, disclosed for one aspect hence also apply to the other aspects.

The invention shall not be limited to the shown embodiments and examples. While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is to be understood that various alternatives to the embodiments described herein can be employed in practicing the disclosure.

It is to be understood that every embodiment of the disclosure can optionally be combined with any one or more of the other embodiments described herein.

It is to be understood that each component, compound, particle, or parameter disclosed herein is to be interpreted as being disclosed for use alone or in combination with one or more of each and every other component, compound, or parameter disclosed herein. It is further to be understood that each amount/value or range of amounts/values for each component, compound, or parameter disclosed herein is to be interpreted as also being disclosed in combination with each amount/value or range of amounts/values disclosed for any other component(s), compound(s), or parameter(s) disclosed herein, and that any combination of amounts/values or ranges of amounts/values for two or more component(s), compound(s), or parameter(s) disclosed herein are thus also disclosed in combination with each other for the purposes of this description. Any and all features described herein, and combinations of such features, are included within the scope of the present invention provided that the features are not mutually inconsistent.

### EXAMPLES

### Abbreviations

AM active muscle
AST aspartate aminotransferase
CMJ countermovement jump
EMG electromyography
MIF maximal isometric force
MDF median frequency
MPF mean propulsive force
MPP mean propulsive power
MPV mean propulsive velocity
OB overall body
PC phenylcapsaicyn
PLA placebo
PRS perceived recovery status
RFD rate of force development
RMS root mean square
RM repetition maximum
RPE ratings of perceived exertion
VL velocity loss
VLA vastus lateralis
VM vastus medialis

### Example 1: Evaluation of effects of Phenylcapsaicyn on Resistance Training Performance, Muscle Damage, Protein Breakdown, Metabolic Response, Ratings of Perceived Exertion and Recovery

### A Randomized, Triple-Blinded, Placebo-Controlled, Crossover Trial.

### Introduction

The objective of the study was to evaluate the ergogenic effects of phenylcapsaicyn (PC), including exploring the effects on resistance training performance, muscle damage and metabolic responses. Therefore, the effects of different doses of PC on lower limbs performance in the squat exercise under velocity-based control, metabolic responses to exercise, acute biochemical muscle damage and protein breakdown, rating of perceived exertion overall body (RPE-OB) and of active muscle (RPE-AM) and perceived recovery in resistance trained men, were explored. More specifically, the aim of this study was to explore the effects of a low dose (LD) of 0.625 mg and a high dose (HD) of 2.5 mg of phenylcapsaicyn (PC) on full squat velocity performance, active muscle (AM) and overall body (OB) ratings of perceived exertion (RPE), muscle damage, protein breakdown, metabolic response and 24 hours recovery in comparison to placebo (PLA).

### Materials and Methods

25 healthy men (age=21.00±2.15 years, body mass=76.54±9.50 kg, height=176.39±7.47 cm, squat 1-repetition maximum [1 RM] normalized to body mass=1.66±0.22 kg) enrolled voluntarily to participate in this randomized, triple-blinded, placebo-controlled, crossover trial.

All participants were resistance trained men with at least 2 years of experience (range=2-5 years). Before the first session, all participants had completed other studies where squat testing was performed in the previous 6 months. All participants were tested for squat strength and anthropometric measurements (i.e., body mass and height) 1 week before the beginning of the study. Exclusion criteria comprised cardiovascular, neurological, physical and/or metabolic disorders which may compromised the primary outcomes.

Participants attended the laboratory twice per week for a total study duration of 3 weeks. Thus, participants undertook a main session and a 24-hours reminder session for each of the 3 supplementation conditions. Each condition included 6 capillary blood extractions, a warm-up, a full squat testing protocol and a 24-hours recovery and muscle damage reminder session. For ensuring a blinding procedure, supplements and placebo were encapsulated and numbered-labelled packaged (Life Pro Nutrition industries, Madrid, Spain) by an independent third-part researcher (i.e., not involved in the study). Packages and capsules were indistinguishable and their content was only revealed after a blinded not-involved in data collection researcher performed the statistical analyses.

Acute oral ingestion of each randomized condition was performed 45 minutes prior to physical testing of the first session of each week. Participants were encouraged to freely select 1 capsule of the assigned numbered package under a researcher supervision. Participants were only allowed to consume the selected capsule with water.

### Supplementation:

Capsules contained either a 0.625 mg low dose of PC (LD), a 2.5 mg high dose (HD) of PC (Axivite, Malmö, Sweden) or a placebo (PLA) composed of maltodextrin and excipients. PC and PLA were identical in appearance, taste and smell. According to EFSA, both doses are considered in the safety range proposed by its expert panel judgement.

### Blood Testing:

Blood samples were extracted and analyzed in 4 different times for each week. Prior to the beginning of each main session, baseline capillary lactate, blood urea an aspartate aminotransferase (AST) samples were collected from the index fingertip of each participant. Timeline for each biomarker of this study was chosen due to suitability and reproducibility according to previous data. Lactate Pro 2 LT-1730 (Arkray, Kyoto, Japan) was used for lactate measurements as it has been shown previously as reliable throughout the physiological range of 1.0-18.00 mmol. Post-test lactate was approached 90 seconds after the last squat set as metabolic indicator of the exercise intensity. Urea and AST were tested with 28.5-31.5 μl blood samples using automatic reflectance photometry (Reflotron, Roche, Boheringer Mannhein, Germany) as whole-body protein breakdown and muscle damage measurement respectively. For the determination of urea and AST, heparinized capillary tubes, pipettes and the manufacturer reagent strips were used immediately after each extraction. Accordingly, post-tests extractions and analyses were performed 40 minutes after the last squat set for urea and before starting the 24 hours reminder session for AST.

### Resistance Training Protocol

A full squat resistance protocol was performed to address mechanical responses to acute oral PC supplementation 40 minutes after the ingestion of the assigned weekly condition in the first session and after blood testing in both sessions. Before the beginning of the study, each participant undertook an initial test with increasing loads for the individual determination of estimated 1-RM and the load-velocity relationship in the full squat exercise. Load-velocity relationships were established after measuring mean propulsive velocity (MPV) for each repetition on a Smith machine with no counterweight mechanism (Multipower Fitness Line, Peroga, Murcia, Spain). MPV was directly measured with a linear velocity transducer (T-Force System, Ergotech, Murcia, Spain) attached perpendicularly to the barbell. MPV values were obtained as fastest, mean and slowest for the three sets and the fastest for the warm-up set.

### Full SQUAT Protocol

The full squat protocol consisted of 3 sets of the daily 70% RM for 8 repetitions. According to warm-up sets and individual load-velocity relationships, 70% RM load was daily established for each participant. This load was chosen because of this RM percentage may involve a submaximal non-extenuating (i.e., without reaching muscle failure) high effort for the selected repetitions. Moreover, as the same repetitions were performed for each set and the absolute load was fixed, the real relative intensity was incremental. As MPV and consequently the percentage of velocity loss (% VL) are indicators of neuromuscular fatigue, 2 repetitions of the 60% RM load were performed 3 minutes and 24 hours after the last set of the 3×8 protocol. Velocity values were treated as the slowest, mean and fastest obtained for each set. The % of mean and maximal velocity loss were also reported.

### Perceived Exertion and Perceived Recovery Status (PRS) Assessment:

Subjective fatigue and recovery assessment was conducted using the perceived recovery status (PRS), RPE-OB and active muscle RPE (RPE-AM) scales. PRS, RPE-AM and RPE-OB are subjective recovery and fatigue status diagrams where cut-off points are fixed from 0 to 10. In PRS, the perceived recovery is set between “very poorly recovered/extremely tired” (value 0) and “very well recovered/highly energetic” (value 10). PRS was explained and evaluated before the start of the post 24 hours follow-up for each condition. Fatigue was using RPE-AM and RPE-OB immediately after each 3×8 set. RPE scales were explained after the 60% RM load and before the 3×8% protocol. Maximum perceived exertion was set on the value 10 which corresponds to reaching exhaustion and non-exertion is represented with value 0. RPE-AM was set up as the local perceived exertion of the quadriceps and RPE-OB as the traditional general perceived exertion of the whole body. Both validated scales were printed and participants were able to visualize each one when they required them.

### Statistical Analysis

Data are presented as means and standard deviations (Mean±SD). The normal distribution of the variables (Shapiro-Wilk test) and the homogeneity of the variances (Levene's test) were tested for each variable (p>0.05). Two-way repeated measures analysis of variance (ANOVA) (condition×time) was used to explore the effect of the interventions (LD, HD, PLA) along the time on the magnitude of each dependent biochemical and perceptual variable. Bonferroni post-hoc comparison was performed when ANOVA significancy was reached. One-way repeated measures ANOVA were used to compared weight and velocity analyses. The Greenhouse-Geisser correction was applied when the Mauchly's sphericity test was significant (p≤0.05). The Cohen's d effect size (ES) with 95% confidence intervals was calculated to evaluate the magnitude of the differences using the following scale: negligible (<0.20), small (0.20-0.49), moderate (0.50-0.79), and large (≥0.80) [31]. If non-parametric data was examined, Friedman and Wilcoxon were used instead. Statistical analyses were performed using the software package SPSS (IBM SPSS version 25.0, Chicago, IL, USA). Statistical significance was set at p≤0.05.

### Results and Discussion Blood Testing

Two-way repeated measures ANOVAs reported significant differences of lactate and urea absolute values for time (pre-post comparisons) (F=269.62, p<0.001) but not for condition (F=1.49, p=0.23) or condition×time interaction (F=0.94, p=0.34) (Table 1). For AST, Friedman test revealed no significant differences between conditions in pre values (p=0.771), showing similar baseline levels.

However, significant differences between conditions were found for aspartate aminotransferase (AST) post values (p=0.029) (Table 1). Post-hoc Wilcoxon test revealed significant higher post levels of AST for PLA compared to HD (p=0.021). Friedman test revealed significant differences between conditions for the percentage of change for urea values (p=0.042). Post-hoc Wilcoxon tests revealed significant higher percentage of change for LID compared to PLA (p=0.026) and compared to HD (p=0.037).

TABLE 1
Absolute and percentual comparison of the biochemical blood tests between the different conditions.

| | | | | | ANOVA | | |
|---|---|---|---|---|---|---|---|
| | | | Condition | | | | Condition × |
| Variable | Time | PLA | LD | HD | Condition | Time | time |
| Lactate | Pre | 1.88 ± 0.59 | 1.70 ± 0.44 | 1.60 ± 0.42 | F = 1.49 | F = 269.62 | F = 0.94 |
| (mmol/L) | Post | 12.37 ± 3.47 | 12.70 ± 4.51 | 11.54 ± 2.81 | p = 0.23 | p = <0.001* | p = 0.34 |
| Urea | Pre | 31.98 ± 11.55 | 29.77 ± 10.67 | 30.31 ± 10.41 | F = 0.11 | F = 32.44 | F = 0.94 |
| (mg/dl) | Post | 38.93 ± 17.36 | 40.77 ± 14.17 | 37.48 ± 18.18 | p = 0.89 | p = <0.001* | p = 0.39 |

| | | | | | |
|---|---|---|---|---|---|
| AST | Pre | 18.62 ± 9.92 | 17.40 ± 11.25 | 16.37 ± 11.29 | Pre; p^{#} = 0.771 |
| (U/L) | Post | 29.55 ± 8.57 | 27.06 ± 14.66 | 22.39 ± 10.86 | Post; p^{#} = 0.029* |
| Lactate | | 596.05 ± 307.30 | 663.97 ± 352.36 | 653.16 ± 307.31 | p^{#} = 0.698 |
| (% change) | | | | | |
| Urea | | 12.40 ± 23.56 | 24.50 ± 23.47 | 9.29 ± 28.46 | p^{#} = 0.042* |
| (% change) | | | | | |
| AST | | 166.27 ± 236.49 | 119.62 ± 166.08 | 110.21 ± 167.16 | p^{#} = 0.167 |
| (% change) | | | | | |

Mean ± standard deviation.
AST, Aspartate aminotransferase; PLA, Placebo; HD, High dose; LD, Low dose.
Pre; measurements; Taken each week before the first session.
Post; Taken 90 seconds after squat testing for lactate, 40 min for urea and 24 hours for AST.
*Significant difference (p ≤ 0.05).
^{#}non-parametric outcome.

### Resistance Training and Movement Velocity

One-way repeated measures ANOVAs reported no significant differences between conditions for weight (F=0.171, p=0.843). On the other hand, the one-way repeated measures ANOVAs of movement velocity outcomes revealed significant effects for 60% load fastest velocity, fastest velocity, mean velocity and maximal velocity loss (p range=<0.001 to 0.05) (Table 2).

Post-hoc Bonferroni reported significant differences between LD and PLA for 60% fastest velocity (p=0.05) and between LD and HD for the fastest velocity of the sets (p=0.009). Bonferroni post-hoc comparisons revealed significant differences between HD and LD for maximal velocity loss (p=0.008) and it almost was achieved between HD and PLA for the mean velocity variable (p=0.06). The magnitude of the differences between the different conditions ranged from negligible to large (Table 3).

TABLE 2
Comparison of the mechanical outcomes of the
study between the different conditions.

| Variable | PLA | LD | HD | ANOVA |
|---|---|---|---|---|
| Weight (kg) | 87.04 ± 14.89 | 87.69 ± 15.87 | 87.57 ± 14.69 | F = 2.95, p = |
| | | | | 0.63 |
| Repetitions | 23.84 ± 0.554 | 24 ± 0.00 | 24 ± 0.00 | F =#, p =# |
| 60% fastest-V | 0.93 ± 0.06 | 0.97 ± 0.07 | 0.96 ± 0.07 | F = 4.78, p = |
| (m · s⁻¹) | | | | 0.014* |
| Fastest-V (m · | 0.78 ± 0.04 | 0.87 ± 0.03 | 0.80 ± 0.06 | F = 103.50, |
| s⁻¹) | | | | p = <0.001* |
| Mean-Vₜ (m · s⁻¹) | 0.59 ± 0.05 | 0.61 ± 0.07 | 0.64 ± 0.07 | F = 4.14, p = |
| | | | | 0.023* |
| Slowest-Vₜ | 0.43 ± 0.10 | 0.45 ± 0.09 | 0.49 ± 0.09 | F = 2.69, p = |
| (m · s⁻¹) | | | | 0.08 |
| MeanLoss-V | 31.18 ± 9.87 | 31.68 ± 9.34 | 27.73 ± 8.79 | F = 2.79, p = |
| (m · s⁻¹) | | | | 0.073 |
| MaxLoss-V | 36.86 ± 10.76 | 37.96 ± 10.59 | 32.79 ± 8.27 | F = 0.33, p = |
| (m · s⁻¹) | | | | 0.05* |

Mean ± standard deviation. Repetitions, Repetitions performed in the protocol; 60% fastest-V, highest velocity measured in the 60% sets; fastest-V, highest velocity measured in the 3 sets; mean-V, mean velocity of all repetitions during the 3 sets; Slowest-V,
Slowest velocity measured in the 3 sets; MeanLoss-V, mean percent loss in velocity from the fastest to the slowest repetition over the 3 sets; MaxLoss-V, maximum percent loss in velocity from the fastest to the slowest repetition over the 3 sets; PLA, Placebo; LD, Low dose; HD, High dose;
*Significant difference (p ≤ 0.05);
#value not defined.

TABLE 3
Cohen's d effect size (ES) with 95% confidence intervals
(CI) comparing mechanical outcomes between conditions.

| | LD vs. PLA | | HD vs. PLA | | LD vs. HD | |
|---|---|---|---|---|---|---|
| Weight (kg) | 0.04 | (−0.12, 0.21) | 0.03 | (−0.14, 0.21) | 0.01 | (−0.12, −0.14) |
| 60% fastest-V (m · | 0.57 | (0.11, 1.03) | 0.34 | (−0.05, 0.57) | 0.22 | (−0.07, 0.51) |
| s⁻¹) | | | | | | |
| Fastest-V (m · s⁻¹) | 2.22 | (1.76, 2.67) | 0.41 | (0.00, 0.82) | 1.17 | (0.65, 1.17) |
| Mean-Vt (m · s⁻¹) | 0.08 | (−0.36, 0.51) | 0.53 | (0.08, 0.99) | −0.37 | (−0.65, −0.08) |
| Slowest-Vt (m · s⁻¹) | 0.16 | (−0.36, 0.069) | 0.53 | (−0.06, 1.13) | −0.37 | (−0.65, −0.08) |
| MeanLoss-V (m · | 0.09 | (−0.26, 0.44) | −0.33 | (−0.80, 0.14) | 0.38 | (0.06, 0.69) |
| s⁻¹) | | | | | | |

Mean ± standard deviation. Repetitions, Repetitions performed in the protocol; 60% fastest-V, highest velocity measured in the 60% sets; fastest-V, highest velocity measured in the 3 sets; mean-V, mean velocity of all repetitions during the 3 sets; Slowest-V, Slowest velocity measured in the 3 sets; MeanLoss-V, mean percent loss in velocity from the fastest to the slowest repetition over the 3 sets; MaxLoss-V, maximum percent loss in velocity from the fastest to the slowest repetition over the 3 sets; PLA, Placebo; LD, Low dose; HD, High dose. A positive ES indicates a higher value for HD compared to PLA, LD compared to PLA, and LD compared to HD.

### Perceived Exertion (RPE) and Perceived Recovery Status (PRS) Assessments

Two-way repeated measures ANOVAs for RPE-OB reported significant differences for time (F=49.00, p<0.001) but not for condition (F=2.77, p=0.07) or condition×time interaction (F=1.339, p=0.261) (Table 1).

Bonferroni post-hoc analyses revealed no significant differences. For RPE-AM (local RPE in quadriceps), both condition (F=9.19, p<0.001) and time (F=36.154, p<0.001) reached significant differences but not condition×time interaction (F=0.553, p=0.697). Bonferroni post-hoc analyses showed significant differences for all times comparisons (p range=<0.001 to 0.002) and for comparisons between PLA and HD (p=0.004) and between HD and LD (p=0.016). On the other hand, one-way repeated measures ANOVAs found no differences between conditions for PRS (F=0.698, p=0.463). Results and descriptive values as mean±SD and individual variability are depicted in FIG. 1. This figure includes graphs a), b) and c) wherein Individual (points) and mean (bars) values of:
(a) RPE-OB, Overall body rating of perceived exertion;
(b) RPE-AM, Active muscle rating of perceived exertion;
(c) PRS, Perceived recovery status,
for the different supplementation conditions (PLA, HD, LD). PC, Phenylcapsaicyn; S1/2/3, Set1/2/3 (* p ANOVA<0.05; #p Bonferroni≤0.05).

In summary, the phenylcapsaicyn high dose (HD) provided significant differences for rated perceived exertion active muscle (RPE-AM), aspartate aminotransferase (AST), % of urea change and the mean propulsive velocity (MPV) of the slowest repetitions and the maximal velocity loss compared to LD and PLA. Post-hoc analyses revealed significant differences between HD and PLA for AST, % urea change, RPE-AM and maximal velocity loss; for % of urea change between HD and LD; and for % of change of urea and 60% fastest velocity between PLA and LD. LD elicited the fastest repetition compared to HD (p≤0.05). The magnitude of the differences between conditions ranged from negligible to large. Hence, phenylcapsaicyn may favourably impact performance, rated perceived exertion of active muscle (RPE-AM), muscle damage, protein breakdown and recovery status compared to placebo.

A plausible mechanism linking force application, muscle damage, protein breakdown and peripheral perceived exertion may provide an explanation of the ergogenic effects of PC on resistance squat exercise. This ergogenic effect may only appear after a “dose” threshold is reached. Collectively, the findings from this study suggest that a 2.5 mg dose of PC provides a plausible ergogenic effect on sport performance, muscle damage, protein breakdown and peripheral perceived exertion in comparison to PLA and a low dose of 0.625 mg. Moreover, a 0.625 dose of PC may be able to increase low-fatigue mechanical performance compared to PLA.

### CONCLUSION

The results of the present study suggest that a high dose (2.5 mg) of phenylcapsaicyn supplementation ingested 45 minutes prior to exercise may increase performance on the squat exercise and reduce muscle damage, protein breakdown and peripheral quadriceps perceived exertion on trained subjects in comparison to a low dose (0.625 mg) and placebo. The main findings were that phenylcapsaicyn high dose (HD) reduced perceived exertion for the active muscle, RPE-AM, enhanced mechanical performance, and exhibited lower muscle damage and percentual change of protein breakdown values, in comparison to the placebo (PLA) and the low dose of phenylcapsaicyn (LD).

Phenylcapsaicyn in the low dose (LD, 0.625 mg), in the other hand, was effective inducing significant differences in the fastest repetitions of both loads (i.e., in the less strenuous repetitions) in comparison to HD and PLA. The low dose may increase mechanical performance in low fatigue tasks but not when exercise is performed near exhaustion.

Collectively, the ergogenic effects of phenylcapsaicyn on performance outcomes were verified for HD and partially for LD. Furthermore, HD was effective in reducing muscle damage and protein breakdown. Therefore, the results of the present study confirm a plausible ergogenic effect of phenylcapsaicyn.

### Example 2: Evaluation of Effects of Phenylcapsaicyn on Neuromuscular Activity and Mechanical Performance in Dynamic and Isometric Exercises

### A Randomized, Triple-Blinded, Crossover, Placebo-Controlled Trial.

### Introduction

The objective of the study was to examine the effects of phenylcapsaicyn (PC) supplementation on mechanical performance and neuromuscular activity. Mechanical performance was evaluated in the dynamic full squat exercise, countermovement jump (CMJ) and isometric squat. Neuromuscular fatigue after resistance training was approached calculating the differences in mechanical performance of a reliable variable as countermovement jump (CMJ) height loss or linear velocity loss with a matched load.

Therefore, the aim of this study was to examine the effects of two different doses of phenylcapsaicyn (PC, LD and HD) on isometric and dynamic performance, neural excitability, fatigue and recovery throughout the CMJ and full squat exercises. For this purpose, a randomized, triple-blinded, placebo-controlled crossover trial with 2 sessions per condition was performed. It was hypothesized that PC may acutely increase velocity, force and power production in the squat exercise, neural excitability and CMJ height. However, due to the improvements on performance, a higher degree of fatigue and a detrimental effect on mechanical recovery in the post-test measurements were also expected.

### Materials and Methods

### Experimental Approach to the Problem

This study was conceived as a randomized, triple-blinded, crossover, placebo-controlled trial. Subjects completed 3 experimental conditions, each one composed of a main session and a 24 hours second session. Subjects performed each session at the same individual time of the day under stable environmental conditions (22-24° C. and 55% humidity). Two weeks prior to the beginning of the study, subjects were anthropometrical (body mass and height), 1 RM in squat and load-velocity relationship tested. On the following 3 weeks, subjects randomly ingested either placebo (PLA) or a low (LD) or high (HD) dose of PC prior to the first weekly session. Then, subjects warmed-up and performed CMJ and squat tests. In the second session subjects did not consume any of the conditions. Thus, after the warm-up, they were directly evaluated for CMJ and squat tests again. Electromyographical assessment of each session was recorded while subjects were performing the squat tests.

### Subjects

Twenty-five healthy men (age=21.69±3.65 years, body mass=77.43±9.09 kg, height=176.74±7.20 cm, squat 1-repetition maximum [1 RM]=125.62±21.01 kg, RM normalized to body mass=1.64±0.22 kg) with at least 2 years of experience on resistance training (range=2-5 years) were recruited for this study. If subjects suffered from any cardiovascular, muscular, neurological, and/or metabolic disorder they were directly excluded. Once subjects were informed about the aim of the study, procedures and possible risks, subjects freely signed the information consent sheet. The present research was approved by the Research Ethics Committee of Pablo of Olavide University in accordance to the tenets of the Declaration of Helsinki. Each condition was established under the safety ranges proposed by European Food Safety Authority (EFSA) expert panel (10).

Participants were asked not to ingest stimulants (e.g., caffeine) or other ergogenic aids previously to each session, not to perform strenuous physical activity and not to modify their dietary intake 2 days before the tests. During the 3 weeks of the study, 2 participants withdrew of the study. One of them due to injury and the other because of missing the last session.

### Supplementation Procedures

Supplements and placebo were prepared and packaged by a non-involved researcher in independent installations (Life Pro Nutrition industries, Madrid, Spain). For ensuring blinding, each package was encoded with a number from 1 to 3. Packages and capsules were identical in appearance, color and taste and their content were only revealed after statistical analyses were finished. Capsules composition included either of:
a maltodextrin and excipients placebo with a red dye (PLA),
a high dose (HD) of PC (Axivite, Malmö, Sweeden) of 2.5 mg, or
a low dose (LD) of PC of 0.625 mg.

Randomization and crossover were performed 2 weeks prior to the beginning of the study. For reducing possible bias, a third-part researcher assigned subjects to each condition with Research Randomizer website (www.randomizer.org). Each subject consumed 1 condition per week along the 3 total weeks of the study. PC doses or placebo were ingested 45 minutes previous to the first exercise session. Researchers encourage subjects to freely selected a capsule from the daily assigned condition package. Capsules were taken with water under the supervision of at least 1 researcher.

### Electromyography (EMG)

Before EMG recording, each subject was shaved and drawn with a black permanent marker for ensuring consistency in electrodes position across conditions (30). Surface EMG electrodes were placed over vastus medialis (VM) and vastus lateralis (VLA) of the right leg. EMG signals were evaluated for 60% and 70% repetition maximum (RM) sets and for isometric tests in both sessions. EMG signals were recorded continuously with a bipolar, parallel-bar surface electromyographic wireless Trigno™ sensor. Baseline noise was established on <5 μV peak-to-peak and sampling rate was 1926 Hz. EMG system was set on an inter-electrode distance of 10 mm, common mode rejection ratio>80 dB, and bandwidth filter between 20 and 450 Hz±10% (Delsys Inc, MA, USA). Data was stored using EMG works Acquisition software (Delsys Inc, MA, USA). For each measure, the median frequency (MDF) and root mean square (RMS) where individually calculated for VM and VLA as excitatory muscle activity assessments. All the outcomes measured were recorded for each individual repetition (over sliding windows of 500 ms with an overlap of 499 ms) and averaged for further analysis for the dynamic and isometric tests. For ensuring inter-conditions reliability, data were normalized under the daily maximal value of the first isometric signal. Thus, EMG values were expressed as a percentage of the maximal daily value obtained.

### Resistance Training Protocol

### Warm-Up

A standardized warm-up was performed 30 minutes after capsule ingestion in the first session and immediately after subjects arrived to the laboratory in the second session. All subjects were inspected regarding EMG marks before the warm-up. The warm-up consisted of (1) 5 minutes of submaximal continuous running at 9 km-h-1, (II) 3 sets of 10 repetitions of bodyweight squat, 3 progressive CMJs, (III) 2 maximal CMJs repetitions and 3 sets of 2 repetitions with the 40%, 50% and 60% of 1 RM in the full squat exercise. Resting between full squat sets was fixed in 2 minutes.

### Countermovement Jump (CMJ) Test

CMJ height was determined using an infrared timing system (OptojumpNext,Microgate, Bolzano, Italy) (31). Subjects were instructed in performing CMJs with their arms akimbo during eccentric and concentric phases. Accordingly, CMJ technique was established as a 90° of knee flexion, followed by a maximal vertical jump. For each trial, landing was required to be in an upright position without knees bending until the movement was completed. For each measurement, subjects were required to perform 2 trials separated by 10 seconds and the mean value was calculated for further analysis. If CMJ difference was greater than 2 cm between trials, a third measurement was required and the 2 nearest values were averaged. CMJ tests were performed twice: before the specific squat warm-up in both sessions and after the last 70% RM set in the first session.

### Isometric Squat Test

Isometric squat tests were performed on 90° of knee flexion position (180°=full extension) for elucidating the effects of PC on maximal isometric force (MIF) and maximal rate of force development (RFDmax). For each condition, 3 isometric tests were performed: (I) 2 minutes before and (II) 3 minutes after the 70% RM sets in the first session and (III) 2 minutes after the 60% RM set in the second session. For this purpose, a Smith machine with customizable height supports was equipped with an 80×80-cm dynamometric platform (FP-500, Ergotech, Murcia, Spain). Subjects were instructed to push with their legs against the floor of the platform as hard as possible after the cue “ready, set, go!”. Subjects were required to execute two 5-s trials separated by 1 min of rest per test. External forces of each trial were collected at a sampling rate of 1000 Hz and processed with specific software (T-Force System, Ergotech, Murcia, Spain). For RFDmax assessment, the maximum slope in the force-time curve in 20-ms time intervals was selected. Furthermore, as RFD data was represented for different discriminable time gaps, RFD was calculated for the 0-50, 0-100, 0-150, 0-200, 0-400 ms intervals. RFD and MIF outcomes were both averaged for further analysis.

### Dynamic Squat Protocol

For the dynamic squat evaluation, the last set of the squat warm-up (1×2×60% RM), 3 sets of 8 repetitions with 70% RM and another 1×2×60% RM after the 60% RM load were recorded during the first session for each condition. In the second session subjects were required to perform a third 1×2×60% RM as a comparator of the degree of mechanical fatigue across time. The mean propulsive values of velocity (MPV), force (MPF) power (MPP) and velocity loss values were acquired from the force platform synchronized with a linear velocity transducer (T-Force System, Ergotech, Murcia, Spain) attached perpendicularly to the barbell. As individual force-load relationships were calculated in the first visit, the real % of RM load was daily adapted. Resting between sets was fixed on 2 minutes for ensuring a relative incremental exertion effect of the tests across time.

### Statistical Analysis

Sample size calculation was performed using the G* POWER software (Heinrich-Heine-Universitat Dusseldorf, Germany) with an alpha value of 0.05. Statistical power was fixed in 0.80 and effect size in 0.60 based on the total volume in the squat exercise of previous research (14). At least 21 subjects were required for this study.

Data are presented as means and standard deviations (Mean±SD). The normal distribution of the variables and homoscedasticity were tested with Shapiro-Wilk and Levene's test respectively (p>0.05). Two-way repeated measures analysis of variance (ANOVA) (condition×time) with Bonferroni post-hoc were used to explore the effect of the interventions (LD, HD, PLA) across time on the magnitude of each dependent variable. One-way repeated measures ANOVA were used to compared total volume load. For non-parametric data, Friedman and Wilcoxon tests were used instead. The Greenhouse-Geisser correction was applied when the Mauchly's sphericity test was significant (p s 0.05). Statistical analyses were performed using the software package SPSS (IBM SPSS version 25.0, Chicago, IL, USA). Statistical significance was established at p≤0.05.

### Results

### EMG

Descriptive values and statistical comparisons for EMG outcomes are presented in Table 4 below. Two-way repeated measures ANOVAs revealed significant differences of 60% load RMS in VLA and VM, isometric MDF in VLA and VM, 60% load MDF VLA and training MDF VLA for time (p range=<0.001 to 0.05). On the other hand, significant differences of 60% load RMS VLA and training RMS in VLA and VM were reported for condition (p range=0.039 to 0.05). Post-hoc Bonferroni reported significant differences between PLA and LID for 60% load RMS VLA (p=0.05). Besides, significant differences were found between HD and LID for training RMS VM (p=0.045).

TABLE 4
Electromyographical responses to the three different supplementation conditions.

| | | | | | ANOVA | | |
|---|---|---|---|---|---|---|---|
| | | | Condition | | | | Condition × |
| Variable | Time | PLA | LD | HD | Condition | Time | time |
| Isometric | Post | 90.95 ± 16.07 | 99.88 ± 41.48 | 99.21 ± 50.39 | F = 1.05; | F = 1.46; | F = 0.76; |
| RMS VLA (%) | Post24 | 100.33 ± 29.25 | 126.91 ± 33.50 | 100.25 ± 25.60 | p = 0.35 | p = 0.24 | p = 0.42 |
| Isometric | Post | 117.01 ± 37.88 | 113.38 ± 24.62 | 102.79 ± 32.43 | F = 0.24; | F = 1.24; | F = 0.42; |
| RMS VM (%) | Post24 | 118.27 ± 28.43 | 120.01 ± 58.48 | 118.65 ± 41.54 | p = 0.78 | p = 0.28 | p = 0.65 |
| 60% load | Pre | 122.12 ± 24.90 | 131.16 ± 36.34 | 144.45 ± 50.27 | F = 3.59; | F = 12.42; | F = 1.89; |
| RMS VLA (%) | Post | 97.48 ± 24.60 | 119.38 ± 47.67 | 103.95 ± 49.44 | p = 0.039* | p = 0.001* | p = 0.16 |
| | Post24 | 120.29 ± 36.08 | 149.81 ± 47.00 | 133.16 ± 33.21 | | | |
| 60% load | Pre | 130.81 ± 39.72 | 131.17 ± 29.13 | 120.88 ± 31.08 | F = 0.081; | F = 10.36; | F = 0.612; |
| RMS VM (%) | Post | 114.28 ± 39.81 | 117.18 ± 19.75 | 112.41 ± 42.15 | p = 0.92 | p < 0.001* | p = 0.65 |
| | Post24 | 133.19 ± 39.46 | 133.87 ± 43.81 | 138.30 ± 47.06 | | | |
| Isometric | Post | 94.88 ± 14.37 | 98.12 ± 9.79 | 99.07 ± 13.42 | F = 0.165; | F = 4.909; | F = 2.948; |
| MDF VLA (%) | Post24 | 104.87 ± 19.26 | 97.78 ± 9.79 | 100.39 ± 12.86 | p = 0.84 | p = 0.039* | p = 0.06 |
| Isometric | Post | 98.45 ± 12.33 | 93.75 ± 11.10 | 91.04 ± 14.79 | F = 0.726; | F = 4.121; | F = 1.060; |
| MDF VM (%) | Post24 | 100.28 ± 17.00 | 97.28 ± 13.72 | 101.88 ± 18.22 | p = 0.49 | p = 0.05* | p = 0.34 |
| 60% load | Pre | 93.68 ± 11.38 | 94.20 ± 12.57 | 92.43 ± 15.57 | F = 0.17; | F = 5.45; | F = 0.68; |
| MDF VLA (%) | Post | 89.04 ± 12.03 | 87.66 ± 11.86 | 87.61 ± 11.16 | p = 0.84 | p = 0.009* | p = 0.60 |
| | Post24 | 94.74 ± 18.85 | 89.62 ± 12.28 | 94.85 ± 19.89 | | | |
| 60% load | Pre | 81.41 ± 11.02 | 93.24 ± 12.84 | 87.62 ± 9.55 | F = 1.27; | F = 0.70; | F = 2.36; |
| MDF VM (%) | Post | 82.50 ± 13.84 | 88.27 ± 13.74 | 87.29 ± 15.23 | p = 0.29 | p = 0.50 | p = 0.11 |
| | Post24 | 92.09 ± 16.32 | 88.62 ± 16.06 | 84.97 ± 10.64 | | | |
| Training | Set 1 | 113.55 ± 29.23 | 123.84 ± 34.86 | 121.24 ± 36.40 | F = 3.51; | F = 0.83; | F = 1.37; |
| RMS VLA (%) | Set 2 | 114.37 ± 35.37 | 146.42 ± 47.13 | 119.29 ± 38.40 | p = 0.039* | p = 0.40 | p = 0.26 |
| | Set 3 | 114.56 ± 35.79 | 135.14 ± 53.99 | 107.20 ± 42.77 | | | |
| Training | Set 1 | 118.11 ± 36.79 | 128.39 ± 50.89 | 111.81 ± 31.90 | F = 3.270; | F = 0.247; | F = 0.676; |
| RMS VM (%) | Set 2 | 113.45 ± 34.83 | 132.39 ± 40.98 | 109.58 ± 36.84 | p = 0.05* | p = 0.78 | p = 0.52 |
| | Set 3 | 116.77 ± 37.34 | 138.04 ± 55.37 | 107.44 ± 35.96 | | | |
| Training | Set 1 | 93.80 ± 12.46 | 89.90 ± 10.21 | 90.47 ± 12.71 | F = 0.280; | F = 5.740; | F = 1.327; |
| MDF VLA (%) | Set 2 | 89.75 ± 12.58 | 88.36 ± 11.03 | 89.99 ± 12.70 | p = 0.75 | p = 0.006* | p = 0.26 |
| | Set 3 | 88.81 ± 12.19 | 86.91 ± 9.27 | 89.91 ± 11.39 | | | |
| Training | Set 1 | 84.98 ± 10.91 | 85.63 ± 11.87 | 91.01 ± 15.42 | F = 1.977; | F = 2.315; | F = 0.644; |
| MDF VM (%) | Set 2 | 84.23 ± 11.30 | 84.07 ± 11.93 | 90.42 ± 18.35 | p = 0.15 | p = 0.11 | p =0.63 |
| | Set 3 | 81.85 ± 12.71 | 83.74 ± 9.68 | 90.58 ± 15.25 | | | |

Mean ± standard deviation.
PLA, Placebo; HD, High dose; LD, Low dose; RMS, Root mean square; MDF; Median frequency; VLA, Vastus lateralis; VM, Vastus medialis.
*Significant difference (p ≤ 0.05).

### Countermovement Jump Test

Friedman tests revealed significant differences between conditions for CMJ height in post (p=0.002) and post 24 h values (p=0.003). Post-hoc Wilcoxon tests for CMJ post values reached significant differences for HD compared to both PLA (p=0.009) and LD (p=0.004). On the other hand, for CMJ post 24 h values, significant differences were found when comparing HD to PLA (p=0.02) and LD to PLA (p=0.019). Besides, significant differences were found between conditions for the percentage of change in CMJ from pre to post values (p=0.04) but post-hoc analyses did not reach any significant difference. Descriptive values as mean±SD and individual variability of CMJ comparisons are depicted in FIG. 2. This figure includes graphs a), b) and c) wherein individual (points) and mean (bars) values of (a) CMJ height at pre-test, post-test and CMJ post 24 h, (b) CMJ pre-post-test percentage of difference and (c) CMJ pre-post 24 h percentage of difference for the three different supplementation conditions (PLA, HD, LD) (* p ANOVA≤0.05; #p Bonferroni≤0.05).

### Isometric Squat Test

Descriptive values and statistical comparisons for isometric mechanical outcomes are presented in Table 5. Two-way repeated measures ANOVAs revealed no significant differences for any outcome in time (p range=0.06 to 0.78), condition (p range=0.19 to 0.97) or condition×time interaction (p range=0.09 to 0.89).

TABLE 5
Isometric mechanical responses to the three supplementation conditions.

| | | | | | ANOVA | | |
|---|---|---|---|---|---|---|---|
| | | | Condition | | | | Condition × |
| Variable | Time | PLA | LD | HD | Condition | Time | time |
| MIF (N) | PRE | 1,192.85 ± 421.86 | 1,175.92 ± 370.86 | 1,182.71 ± 429.84 | F = 1.13 | F = 1.94 | F = 0.91 |
| | POST | 1,051.39 ± 316.069 | 1,063.035 ± 265.86 | 1,091.67 ± 331.61 | p = 0.35 | p = 0.16 | p = 0.46 |
| | POST 24 | 1,120.82 ± 440.00 | 1,084.25 ± 375.82 | 1,131.50 ± 420.02 | | | |
| RFDmax | PRE | 4,562.82 ± 1607.70 | 5,041.28 ± 1676.66 | 4,363.17 ± 1234.21 | F = 0.03 | F = 0.30 | F = 1.456 |
| (N · s¹) | POST | 4,395.91 ± 1,689.33 | 3935.83 ± 1,153.02 | 4,678.55 ± 1,658.94 | p = 0.97 | p = 0.78 | p = 0.22 |
| | POST 24 | 4,894.21 ± 1601.29 | 4,075.67 ± 1337.68 | 4,752.17 ± 1585.14 | | | |
| RFD₀₋₅₀ | PRE | 2,262.42 ± 1,189.19 | 2,765.42 ± 1,725.44 | 2,544.07 ± 1,150.29 | F = 1.28 | F = 0.78 | F = 1.55 |
| (N · s¹) | POST | 2,467.75 ± 1,396.78 | 2,335.85 ± 1,408.53 | 2,375.33 ± 1,172.75 | p = 0.29 | p = 0.46 | p = 0.19 |
| | POST 24 | 2,512.35 ± 1,332.47 | 2,006.35 ± 773.62 | 2,190.07 ± 1,249.61 | | | |
| RFD₀₋₁₀₀ | S1 | 2,377.17 ± 1325.04 | 2,921.42 ± 1,353.33 | 2,585.57 ± 1,998.95 | F = 1.73 | F = 1.14 | F = 2.43 |
| (N · s¹) | S2 | 2,039.58 ± 1,283.25 | 2,320.43 ± 1,229.46 | 1,968.46 ± 1,373.43 | p = 0.19 | p = 0.33 | p = 0.10 |
| | S3 | 2,666.17 ± 1,432.51 | 2,129.25 ± 942.03 | 2,377.32 ± 1,196.68 | | | |
| RFD₀₋₁₅₀ | S1 | 2,380.76 ± 1,655.64 | 2,958.42 ± 1,280.93 | 2,635.67 ± 1,075.21 | F = 0.54 | F = 1.29 | F = 2.09 |
| (N · s¹) | S2 | 2,670.78 ± 1,002.99 | 2,388.16 ± 1,184.60 | 2,129.54 ± 1,279.24 | p = 0.58 | p = 0.28 | p = 0.09 |
| | S3 | 2,600.69 ± 1,330.48 | 2,260.47 ± 1,032.89 | 2,450.52 ± 1,275.39 | | | |
| RFD₀₋₂₀₀ | S1 | 2,400.89 ± 1,322.21 | 2,697.29 ± 1,219.14 | 2,456.98 ± 1,052.11 | F = 0.28 | F = 2.56 | F = 1.69 |
| (N · s¹) | S2 | 2,384.10 ± 918.54 | 2,178.30 ± 1,101.08 | 1,973.26 ± 1,214.84 | p = 0.76 | p = 0.10 | p = 0.16 |
| | S3 | 2,295.32 ± 1,430.75 | 2,067.32 ± 978.09 | 2,361.75 ± 1,214.58 | | | |
| RFD₀₋₄₀₀ | S1 | 1,787.15 ± 974.75 | 1,764.86 ± 898.55 | 1,784.80 ± 809.39 | F = 0.47 | F = 3.94 | F = 0.27 |
| (N · s¹) | S2 | 1,652.53 ± 772.03 | 1,489.55 ± 733.99 | 1,521.34 ± 853.94 | p = 0.62 | p = 0.06 | p = 0.89 |
| | S3 | 1,631.75 ± 1,045.07 | 1,610.34 ± 773.99 | 1,664.43 ± 912.04 | | | |

Mean ± standard deviation.
PLA, Placebo; HD, High Dose; LD, Low dose; MIF; Maximal isometric force; RFD, Rate of force development.
*Significant difference (p ≤ 0.05).

### Dynamic Squat Protocol

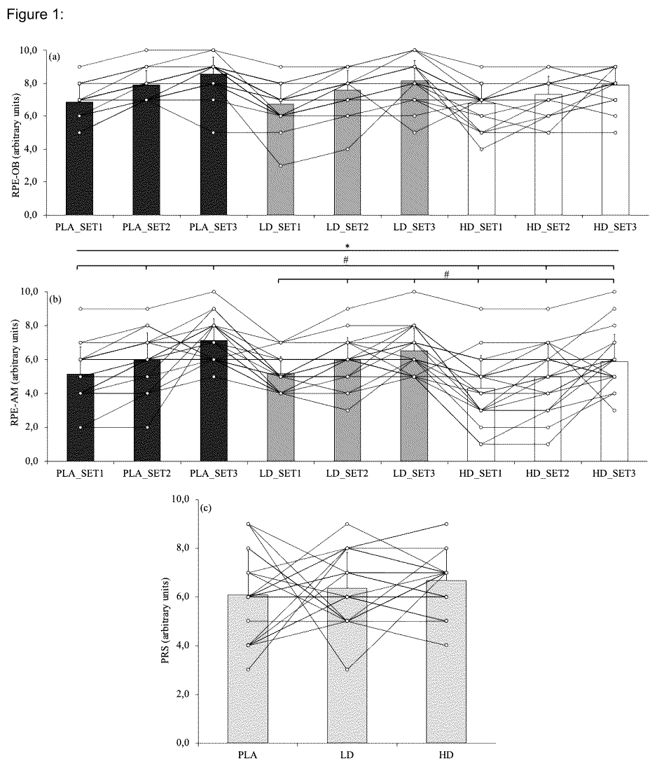

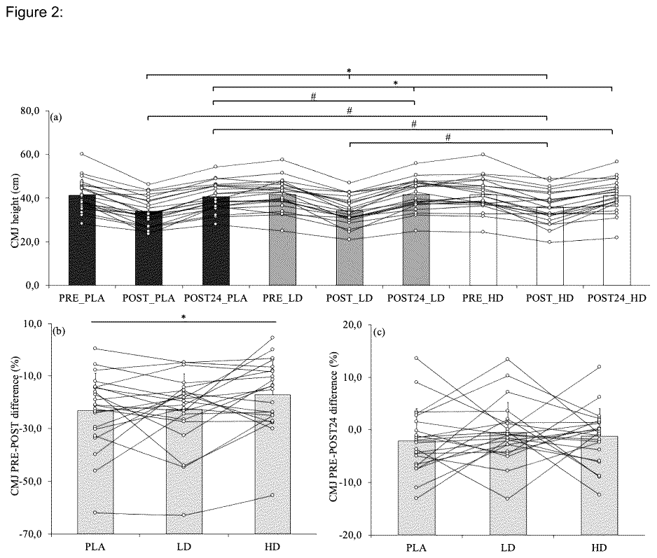

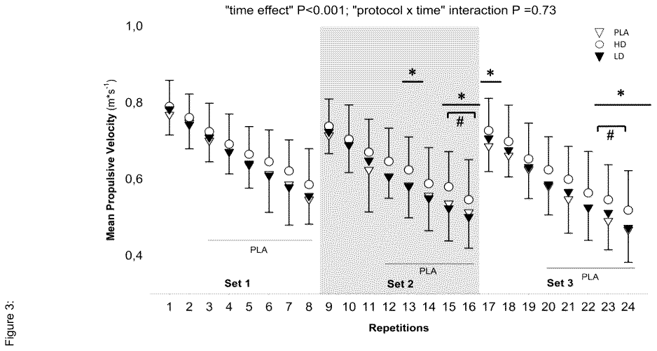

One-way repeated measures ANOVAs reported no significant differences between conditions for total weight lifted (F=1.087, p=0.346). On the other hand, the two-way repeated measures ANOVAs of movement velocity outcomes revealed significant effects for time (p=<0.001) and condition (p range=0.02 to 0.24) but not for condition×time interaction (p range=0.74 to 0.90) (Table 6). Post-hoc Bonferroni for time analyses revealed significant differences between set 1 and 2 for all outcomes (p range=<0.001 to 0.007). However, between set 1 and 3, Bonferroni post-hoc analyses only reported significant differences for mean MPV (p<0.001). For comparisons between set 2 and 3, all movement velocity outcomes reported significant differences (p range=<0.001 to 0.036). Individual intra-set analysis revealed significant differences on repetitions 13, 15,16,17, 23 and 24 (p range=0.027 to 0.04). Post-hoc Bonferroni reported differences between HD and PLA in repetition 23 and for HD and LD in repetitions 15 and 16 (p range=0.03 to 0.04), as shown in FIG. 3, providing the mean propulsive velocity (MPV) in an intra-set comparison of individual repetitions and repetitions across time for the different supplementation conditions (PLA, HD, LD, (* p ANOVA≤0.05; #p Bonferroni≤0.05).

TABLE 6
Dynamic mechanical responses to the three supplementation conditions.

| | | | | | ANOVA | | |
|---|---|---|---|---|---|---|---|
| | | | Condition | | | | Condition × |
| Variable | Time | PLA | LD | HD | Condition | Time | time |
| Total | | 2,089.14 ± 357.56 | 2,014.57 ± 380.94 | 2,0101.71 ± 14.69 | F = 1.087 | | |
| volume | | | | | p = 0.346 | | |
| load (kg) | | | | | | | |
| 60% load | PRE | 0.91 ± 0.07 | 0.93 ± 0.06 | 0.93 ± 0.06 | F = 3.94 | F = 50.89 | F = 0.48 |
| MPV best | POST | 0.77 ± 0.09 | 0.80 ± 0.08 | 0.81 ± 0.12 | p = 0.03* | p = <0.001* | p = 0.74 |
| (m/s) | POST 24 | 0.89 ± 0.07 | 0.93 ± 0.09 | 0.91 ± 0.08 | | | |
| 60% load | PRE | 990.87 ± 162.85 | 950.82 ± 170.05 | 945.34 ± 152.48 | F = 0.61 | F = 2.47 | F = 0.36 |
| MFP best | POST | 924.91 ± 158.99 | 907.38 ± 148.53 | 903.85 ± 155.90 | p = 0.54 | p = 0.09 | p = 0.83 |
| (N) | POST 24 | 951.37 ± 160.79 | 930.60 ± 146.67 | 936.27 ± 149.32 | | | |
| 60% load | PRE | 844.34 ± 173.45 | 822.00 ± 166.47 | 810.71 ± 157.90 | F = 0.92 | F = 23.00 | F = 0.17 |
| MPP best | POST | 691.57 ± 162.32 | 688.32 ± 166.67 | 698.67 ± 180.46 | p = 0.07 | p = <0.001* | p = 0.95 |
| (W) | POST 24 | 790.41 ± 178.79 | 796.70 ± 149.05 | 798.56 ± 170.19 | | | |
| MPV | S1 | 0.77 ± 0.04 | 0.78 ± 0.05 | 0.79 ± 0.06 | F = 1.40 | F = 65.53 | F = 0.01 |
| best (m/s) | S2 | 0.72 ± 0.04 | 0.72 ± 0.07 | 0.74 ± 0.07 | p = 0.24 | p = <0.001* | p = 0.90 |
| | S3 | 0.70 ± 0.05 | 0.71 ± 0.07 | 0.73 ± 0.05 | | | |
| MFP | S1 | 1,030.81 ± 157.73 | 1,040.34 ± 165.74 | 1,016.75 ± 163.05 | F = 0.44 | F = 54.17 | F = 0.068 |
| best (N) | S2 | 1,005.24 ± 170.49 | 1,007.05 ± 176.02 | 994.27 ± 156.37 | p = 0.515 | p = <0.001* | p = 0.797 |
| | S3 | 993.27 ± 172.34 | 1006.04 ± 166.75 | 981.81 ± 145.03 | | | |
| MPP | S1 | 749.01 ± 137.75 | 770.42 ± 151.29 | 744.70 ± 169.44 | F = 0.576 | F = 59.14 | F = 0.90 |
| Best (W) | S2 | 689.28 ± 135.02 | 694.45 ± 160.43 | 701.32 ± 147.81 | p = 0.567 | p = <0.001* | p = 0.46 |
| | S3 | 667.64 ± 161.64 | 681.99 ± 149.20 | 683.86 ± 141.59 | | | |
| MPV | S1 | 0.65 ± 0.04 | 0.66 ± 0.07 | 0.68 ± 0.07 | F = 4.14 | F = 98.13 | F = 0.50 |
| mean (m/s) | S2 | 0.60 ± 0.05 | 0.60 ± 0.07 | 0.64 ± 0.08 | p = 0.02* | p = <0.001* | p = 0.77 |
| | S3 | 0.57 ± 0.06 | 0.58 ± 0.08 | 0.61 ± 0.08 | | | |
| MFP | S1 | 970.36 ± 145.46 | 974.60 ± 162.62 | 962.14 ± 148.59 | F = 0.44 | F = 42.81 | F = 0.110 |
| mean (N) | S2 | 948.51 ± 154.15 | 950.75 ± 161.58 | 942.66 ± 141.94 | p = 0.64 | p = <0.001* | p = 0.979 |
| | S3 | 936.71 ± 152.84 | 941.04 ± 156.67 | 931.41 ± 141.91 | | | |
| MPP | S1 | 619.58 ± 112.77 | 626.70 ± 147.11 | 612.99 ± 145.84 | F = 0.45 | F = 64.39 | F = 1.89 |
| mean (W) | S2 | 561.40 ± 117.83 | 559.89 ± 128.61 | 583.27 ± 128.14 | p = 0.64 | p = <0.001* | p = 0.12 |
| | S3 | 529.28 ± 123.35 | 539.99 ± 126.34 | 548.46 ± 135.58 | | | |
| % VL | S1 | 29.38 ± 8.48 | 29.78 ± 10.90 | 26.62 ± 9.37 | F = 3.73 | F = 5.43 | F = 0.11 |
| | S2 | 29.93 ± 11.87 | 30.91 ± 10.24 | 26.65 ± 11.73 | p = 0.04* | p = 0.008* | p = 0.978 |
| | S3 | 34.24 ± 11.86 | 34.36 ± 12.15 | 29.78 ± 10.90 | | | |

Mean ± standard deviation.
PLA, Placebo; HD, High Dose; LD, Low dose; Mean propulsive velocity; MPF, Mean propulsive force; MPP, Mean propulsive power; % VL, Percentage of velocity loss during a set.
*Significant difference (p ≤ 0.05).

The objective of this study was to explore for the first time the neuromuscular and mechanical responses to a capsaicinoid supplement in dynamic and isometric exercises. The main findings of this research indicate that a low dose of PC may modulate the excitability of motor units of muscles, specifically (i.e., RMS) in vastus lateralis (VLA) and vastus medialis (VM), in comparison to placebo (PLA) and a high dose of PC during dynamic but not during isometric squat exercise. Furthermore, significant differences were revealed for mean propulsive velocity (MPV) and % velocity loss, but not for any other mechanical performance variable as force and power in dynamic or isometric tests. However, the HD condition exhibited higher values in the countermovement jump (CMJ) post and post 24 stages compared to placebo and the lower dose. A positive effect was reported for velocity (MPV), individual repetitions and mechanical recovery outcomes. A dose-response relationship was not established between the different PC doses and the neural output. Therefore, PC supplementation, particularly in the high dose, may attenuate acute and plausibly post-24 mechanical fatigue. These effects on fatigue outcomes may not be mediated by the traditional neural mechanisms proposed in previous literature.

Collectively, an acute oral low dose (LD; 0.625 mg) of PC may increase neural activity during dynamic resistance training and a high dose (HD; 2.5 mg) may reduce acute mechanical fatigue (i.e., lower CMJ height loss and upper MPV values) compared with each other and PLA. Therefore, PC may be a valuable tool for high-volume resistance training workouts.

This study offers valuable insights regarding capsaicinoids supplementation. Acute, phenylcapsaicyn ingestion may be considered as an anti-fatigue ergogenic aid (2.5 mg) for dynamic resistance training sessions when more than 1 exercise is performed. Consistently, mechanical fatigue after a submaximal exercise may be delayed by the composition of the invention.

### REFERENCES

### Example 3 (Prospective): Alternative Compounds of Formula (I)

In order to show that the invention is applicable for a variety of compounds of Formula (I), several compounds may be manufactured and explored for their ergogenic effect.

The following compounds of the invention may e.g. be synthesized and tested: methylcapsaicyn, ethylcapsaicyn, propylcapsaicyn, butylcapsaicyn.

The suggested compounds above may accordingly be studied, e.g. as shown in Example 1 or 2 for phenylcapsaicyn, to evaluate the potential of the compounds as ergogenic aids.

1. A method of enhancing physical performance, said method comprising administering an ergogenic composition in an amount to a subject, said composition comprising a compound of formula (I) 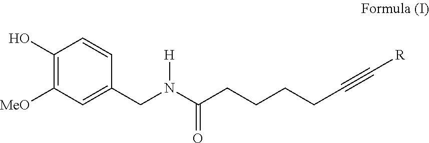
wherein R is alkyl, trifluoromethyl, cycloalkyl, phenyl, halogen, or carbon chain that is straight-chained or branched and optionally further substituted with alkyl, alkenyl, alkynyl, allyl, aryl, alkoxy, aryloxy, alkanoyl, aroyl, amino alkylthio, arylthiol, cyano, cycloalkyl, cycloalkenyl, halo, hydroxy, oxo, nitro, or trifluoromethyl, or tautomers or salts thereof,
wherein said amount is sufficient for enhancing physical performance or promoting accelerated recovery or reducing or delaying mechanical fatigue of said subject.

2. The method of claim 1, wherein R comprises a 1 to 6 carbon atom long chain or R is a phenyl group.

3. The method of claim 1, wherein R is phenyl.

4. The method of claim 1, wherein said administering is oral.

5. (canceled)

6. The method of claim 1, said amount effective for lowering one or more of a level of aspartate aminotransferase (AST), lactate in said subject's blood.

7. The method of claim 1, said enhanced physical performance positively affecting one or more of resistance training performance, mechanical dynamic performance, muscle damage, metabolic response, protein breakdown, neuromuscular fatigue, neural excitability or neuromuscular activity.

8. The method of claim 1, said amount effective for a mechanical anti-fatigue effect on said subject.

9. The method of claim 1, wherein said composition is administered to said subject suffering or recovering from physical weakness, lack of energy, or asthenia.

10. The method of claim 1, wherein said administering to said subject is prior to an exercise.

11. A sport diet supplement composition comprising i) one or more ingredients selected from the group consisting of adaptogens, amino acids, antioxidants, electrolytes, energy boosters and creatine, plus ii) a compound of formula (I): 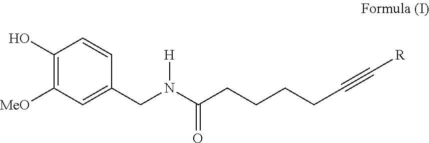
wherein R is alkyl, trifluoromethyl, cycloalkyl, phenyl, halogen, or a carbon chain that is straight-chained or branched and optionally further substituted with alkyl, alkenyl, alkynyl, allyl, aryl, alkoxy, aryloxy, alkanoyl, aroyl, amino alkylthio, arylthio, cyano, cycloalkyl, cycloalkenyl, halo, hydroxy, oxo, nitro, or trifluoromethyl, or tautomers or salts thereof,
provided in a unit dosage form comprising 0.5 to 4.0 mg of said compound of formula (I).

12. The composition of claim 11, said composition formulated as a powder, granulate, tablet, capsule, an aerosol inhalation formulation, a beverage, or a food, such as a bar.

13. The composition of claim 11 wherein R comprises a 1 to 6 carbon atom long chain or a phenyl group.

14. The composition of claim 11 wherein:
said adaptogens are selected from Rhodiola Rosea and Ashwagandha,
said amino acids are selected from Branched-Chain Amino Acids (BCAAs) and L-arginine,
said antioxidants are selected from Vitamins C and E and quercetin,
said electrolytes are selected from potassium, magnesium, and sodium, or
said energy boosters are selected from caffeine and guarana.

15. The composition of claim 11, further comprising caffeine.

16. A method of treatment, said method comprising administering a composition comprising a compound of Formula (I) plus an acceptable carrier to a patient having chronic fatigue syndrome (CFS), myalgic encephalomyelitis (ME) or ME/CFS in an amount sufficient to treat said CFS, ME or ME/CFS, 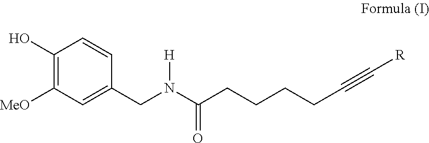
wherein R is alkyl, trifluoromethyl, cycloalkyl, phenyl, halogen, or a carbon chain that is straight-chained or branched and optionally further substituted with alkyl, alkenyl, alkynyl, allyl, aryl, alkoxy, aryloxy, alkanoyl, aroyl, amino alkylthio, arylthio, cyano, cycloalkyl, cycloalkenyl, halo, hydroxy, oxo, nitro, or trifluoromethyl, or tautomers or salts thereof.

17. The method of claim 16, wherein R comprises a 1 to 6 carbon atom long chain or a phenyl group.

18. The method of claim 16, wherein said amount is effective for lowering one or more of a level of aspartate aminotransferase (AST), urea or lactate in said patient's blood.

19. The method of claim 16, wherein said composition further comprises one or more ingredients selected from the group of adaptogens, amino acids, antioxidants, electrolytes, energy boosters and creatine.

20. The method of claim 19, wherein:
said adaptogens are selected from Rhodiola Rosea and Ashwagandha;
said amino acids are selected from Branched-Chain Amino Acids (BCAAs) and L-arginine;
said antioxidants are selected from Vitamins C and E and quercetin;
said electrolytes are selected from potassium, magnesium, and sodium; or
said energy boosters are selected from caffeine and guarana.
